(19) United States
(12) Patent Application Publication
(10) Pub. No.: US 2025/0229098 A1
(43) Pub. Date: Jul. 17, 2025

(54) INTERACTIVE PAIN MANAGEMENT SYSTEM

(71) Applicant: Bruce Coren, West Palm Beach, FL (US)

(72) Inventors: Bruce Coren, West Palm Beach, FL (US); Joseph Costello, West Palm Beach, FL (US)

(21) Appl. No.: 18/412,916

(22) Filed: Jan. 15, 2024

(51) Int. Cl. A61N 5/06; A61N 5/067

(52) U.S. Cl. CPC A61N 5/0613; A61N 5/067

(57) ABSTRACT

An interactive pain management system is provided comprising a laser treatment device and an application executing on the device that receives entry of identification information for a first patient and receives entry of a body region to receive treatment. The system also receives entry of a present pain level experienced in the body region, accesses stored information describing previously reported conditions of the first patient and based on the received entries and the accessed store information, determines a treatment regimen for the first patient. The laser treatment device executes the treatment regimen by manual administration by the first patient or a qualified technician. The system requests entry of the first patient's present condition, the condition comprising one of feeling worse, feeling better, and experiencing no change since an immediately previous treatment. Changes in the condition of the first patient are incorporated into the determination of the treatment regimen.

### CROSS REFERENCE TO RELATED APPLICATIONS

None

### FIELD OF THE DISCLOSURE

The present disclosure is in the field of laser treatment for pain. More particularly, the present disclosure provides a high-powered laser treatment system that makes inquiries of a present patient regarding the patient's present condition, consults stored records about the patient's previous progress and reported conditions, and based thereon develops a treatment regimen for that day using the system.

### BACKGROUND

Musculoskeletal pain affects over 100 million Americans annually at a cost of over $600 billion a year in medical bills, lost productivity and missed work or school. All therapeutic treatments have benefits, but also result in different side effects, risks or complications. Current treatments for musculoskeletal pain include modalities, immobilization, medications, chiropractic care, physical therapy, behavioral management, injections, and surgery.

These approaches have particular associated risk and side effect profiles including peptic ulcers/gastric bleeding, systemic effects (cardiovascular), infections (including epidural abscess), narcotic dependency/addiction, deformities, neurologic deficits, and surgical complications. The natural history of chronic pain is one of increasing dysfunction, impairment and possible disability.

### BRIEF DESCRIPTION OF THE DRAWING

FIG. 1 is an image of an interactive pain management system according to an embodiment of the present disclosure.

### DETAILED DESCRIPTION

An interactive pain management system and method are provided herein which adjusts laser dosage based upon a patient's pain level as well as the patient's response to previous treatments. The system comprises a high-powered laser treatment device and software applications executing thereon that process and store treatment, health, and other records for a plurality of patients. The device identifies and authenticates a patient requesting treatment. Based on the records and on past and present patient inputs, the device develops a treatment regimen for the present session.

Operated by the patient or by a healthcare professional, the device inquires as to the patient's areas of pain in the body that require treatment and levels of pain that day. The device asks if the patient is feeling better, worse, or the same as previously. The device consults its stored records about the patient's previously reported conditions and levels of pain, treatment history, and track record for responding to treatments in the past.

Based on analysis of the patient's input and the data in the patient's records, the device implements a treatment dosage of laser. The patient may administer the treatment him/herself. A professional may administer the treatment.

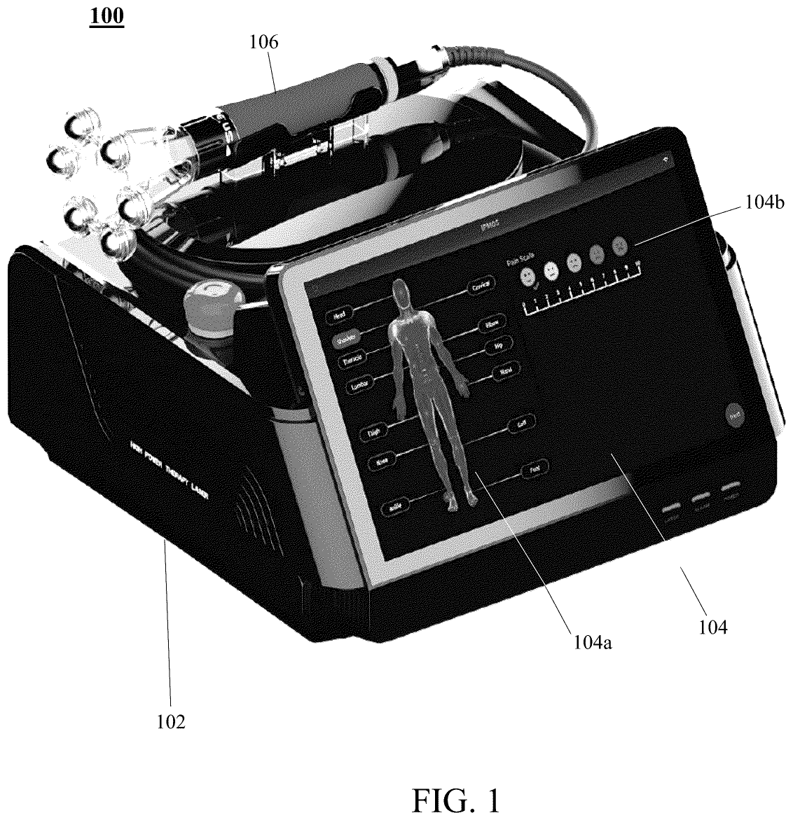

Turning to the FIGURES, FIG. 1 illustrates a system 100 of the interactive pain management system. The system 100 comprises the laser generator 102, a display 104, a body area selection panel 104a, a pain level selection panel 104b, and a laser treatment wand 106. The generator 102 contains functionality for generating the laser energy directed by the application provided herein and sent to the wand 106 for dispensing of treatment.

The generator 102 also contains microprocessors, memory, and storage. The generator 102 also stores the application that processes patient input and calculates the treatment for the present session and stores any data associated with the session. The application is integral to the device and to systems and methods provided herein.

The system 100 may be manually powered on. The patient enters his/her credentials to gain access to his/her account. If the patient is new, the system 100 provides for an account to be created. The system 100, via the display 104, requests the patient to identify the patient's area of pain that day and the level of pain in the afflicted area. The patient makes these entries in the body area selection panel 104a and in the pain level selection panel 104b, respectively.

The system 100 further asks the patient if pain in the afflicted area is better, worse, or unchanged since the previous session. The system 100 also reviews its stored records for the patient to examine how the patient has responded to previous treatments. Based on the patient's present inputs and the system 100 analysis of the inputs and stored data, the system 100 develops a treatment regimen for the present session comprising a dosage of laser and duration of dosage. As noted, the treatment may be administered by the patient him/herself or by a professional. The system saves a record of the present session.

In an embodiment, an interactive pain management system is provided. The system comprises a laser treatment device and an application executing on the device that receives entry of identification information for a first patient, receives entry of a body region of the first patient to receive treatment, and receives entry of a present pain level experienced in the body region. The system also accesses stored information describing previously reported conditions of the first patient and based on the received entries and the accessed store information, determines a treatment regimen for the first patient.

The laser treatment device executes the treatment regimen by manual administration by one of the first patient and by a qualified technician. The device displays a chart of the patient.

The system requests entry of the first patient's present condition, the condition comprising one of feeling worse, feeling better, and experiencing no change since an immediately previous treatment. Changes in the condition of the first patient are incorporated into the determination of the treatment regimen. The system stores records for a plurality of patients and develops treatments for the plurality of patients.

In another embodiment, a method of administering personalized laser treatments to a plurality of patients is provided. The method comprises a laser administration device accessing a datastore describing patients' identifying information, patients' previously reported areas of affliction, patients' previously reported pain levels for the areas, and previous treatments administered to the patients. The method also comprises the device locating in the datastore records for a first patient, the first patient presently seeking treatment by the device. The method also comprises the device determining a treatment regimen for the first patient based on input presently entered by the patient and based on content of the records. The method also comprises the device activating treatment apparatus and administering the determined treatment regimen.

The method also comprises the device requesting of the first patient the input comprising the first patient's present condition, the condition comprising one of feeling worse, feeling better, and experiencing no change since an immediately previous treatment. Changes in the condition of the first patient are incorporated into the determination of the treatment regimen.

The method also comprises the device executing the treatment regimen by manual administration by one of the first patient and a qualified technician. The method also comprises the device displaying a chart of the first patient.

The method also comprises the device considering contents of the chart comprising reports by the first patient of previous reactions to previously received treatment regimens. The method also comprises the device authenticating the first patient before determining and administering the treatment regimen.

In yet another embodiment, a method for administering laser treatments to a plurality of patients is provided. The method comprises a laser treatment device accessing a database describing patients previously having received treatment from the device. The method also comprises the device determining from at least the database that a first patient presently requesting treatment from the device is a previous patient with records stored in the database. The method also comprises the device determining a treatment regimen for the first patient based on patient inputs and based on contents of the records. The method also comprises the device activating treatment apparatus and administering the determined treatment regimen. The method also comprises the device analyzing the patient inputs comprising areas of the body experiencing pain and level of pain in afflicted area. The method also comprises the device requesting of the first patient the input comprising the first patient's present condition, the condition comprising one of feeling worse, feeling better, and experiencing no change since an immediately previous treatment. Changes in the condition of the first patient are incorporated into the determination of the treatment regimen.

The method also comprises the device displaying a chart of the first patient. The method also comprises the device considering contents of the chart comprising reports by the first patient of previous reactions to previously received treatment regimens. The method also comprises the device executing the treatment regimen by manual administration by one of the first patient and a qualified technician.

What is claimed is:

1. An interactive pain management system, comprising:
a laser treatment device; and
an application executing on the device that:
receives entry of identification information for a first patient,
receives entry of a body region of the first patient to receive treatment, receives entry of a present pain level experienced in the body region,
accesses stored information describing previously reported conditions of the first patient, and
based on the received entries and the accessed store information,
determines a treatment regimen for the first patient.

2. The system of claim 1, wherein the laser treatment device executes the treatment regimen by manual administration by one of the first patient and by a qualified technician.

3. The system of claim 1, wherein the device displays a chart of the first patient.

4. The system of claim 1, wherein the system requests entry of the first patient's present condition, the condition comprising one of feeling worse, feeling better, and experiencing no change since an immediately previous treatment.

5. The system of claim 4, wherein changes in the condition of the first patient are incorporated into the determination of the treatment regimen.

6. The system of claim 1, wherein the system stores records for a plurality of patients and develops treatments for the plurality of patients.

7. A method of administering personalized laser treatments to a plurality of patients, comprising:
a laser administration device accessing a datastore describing patients' identifying information, patients' previously reported areas of affliction, patients' previously reported pain levels for the areas, and previous treatments administered to the patients;
the device locating in the datastore records for a first patient, the first patient presently seeking treatment by the device;
the device determining a treatment regimen for the first patient based on input presently entered by the patient and based on content of the records; and
the device activating treatment apparatus and administering the determined treatment regimen.

8. The method of claim 7, further comprising the device requesting of the first patient the input comprising the first patient's present condition, the condition comprising one of feeling worse, feeling better, and experiencing no change since an immediately previous treatment.

9. The method of claim 8, wherein changes in the condition of the first patient are incorporated into the determination of the treatment regimen.

10. The method of claim 7, further comprising the device executing the treatment regimen by manual administration by one of the first patient and a qualified technician.

11. The method of claim 7, further comprising the device displaying a chart of the first patient.

12. The method of claim 11, further comprising the device considering contents of the chart comprising reports by the first patient of previous reactions to previously received treatment regimens.

13. The method of claim 7, further comprising the device authenticating the first patient before determining and administering the treatment regimen.

14. A method for administering laser treatments to a plurality of patients, comprising:
a laser treatment device accessing a database describing patients previously having received treatment from the device;
the device determining from at least the database that a first patient presently requesting treatment from the device is a previous patient with records stored in the database;
the device determining a treatment regimen for the first patient based on patient inputs and based on contents of the records; and
the device activating treatment apparatus and administering the determined treatment regimen.

15. The system of claim 14, further comprising the device analyzing the patient inputs comprising areas of the body experiencing pain and level of pain in an afflicted area.

16. The method of claim 14, wherein the device further comprising the device requesting of the first patient the input comprising the first patient's present condition, the condition comprising one of feeling worse, feeling better, and experiencing no change since an immediately previous treatment.

17. The method of claim 16, wherein changes in the condition of the first patient are incorporated into the determination of the treatment regimen.

18. The method of claim 14, further comprising the device displaying a chart of the first patient.

19. The method of claim 18, further comprising the device considering contents of the chart comprising reports by the first patient of previous reactions to previously received treatment regimens.

20. The method of claim 14, further comprising the device executing the treatment regimen by manual administration by one of the first patient and a qualified technician.
